(19) United States
(12) Patent Application Publication
(10) Pub. No.: US 2022/0185767 A1
(43) Pub. Date: Jun. 16, 2022

(54) NOVEL TERNARY MOLECULAR COMPLEX OF TAMIBAROTENE FOR CANCER STEM CELLS TREATMENT

(71) Applicant: TRANSGENEX NANOBIOTECH, INC., Tampa, FL (US)

(72) Inventors: Mazen HANNA, Tampa, FL (US); Manomi PERERA, Tampa, FL (US); Jiyu YAN, Tampa, FL (US); Andrew HANNA, Lutz, FL (US)

(21) Appl. No.: 17/442,932

(22) PCT Filed: Apr. 1, 2020
(86) PCT No.: PCT/US20/26151

§ 371 Date: Sep. 24, 2021

Related U.S. Application Data
(60) Provisional application No. 62/828,093, filed on Apr. 2, 2019.

(51) Int. Cl. C07C 233/65; C07C 59/245; C07D 235/20; C07C 65/03; C07D 403/10; A61K 31/4178; A61K 45/06; A61K 31/194; A61K 31/4184; A61K 31/192

(52) U.S. Cl. CPC C07C 233/65; C07C 59/245; C07D 235/20; C07C 65/03; C07D 403/10; C07B 2200/13; A61K 45/06; A61K 31/194; A61K 31/4184; A61K 31/192; A61K 31/4178

(57) ABSTRACT

Preparation and characterization of novel tamibarotene forms suitable for pharmaceutical compositions in drug delivery systems for the treatment of cancer stem cells. The tamibarotene compositions can be used for the safe and effective treatment of human diseases including a variety of cancers, cancer stem cells, drug resistant cancers, and used as a radio sensitizer.

### CROSS-REFERENCE TO RELATED APPLICATIONS

This application claims priority to U.S. Provisional Application No. 62/828,093, filed Apr. 2, 2019, the disclosure of which is incorporated herein by reference.

### FIELD OF THE INVENTION

This disclosure pertains to novel crystalline forms of tamibarotene and pharmaceutical compositions comprising the same. The tamibarotene compositions can be used for the safe and effective treatment of human diseases including a variety of cancers, cancer stem cells, drug resistant cancers, and used as a radio sensitizer. The novel forms include but are not limited to cocrystals, salts, solvates of salts, and mixtures thereof. Methods for the preparation of and pharmaceutical compositions suitable for drug delivery systems that include one or more of these new forms are also disclosed.

### BACKGROUND OF THE INVENTION

Tamibarotene is a synthetic retinoid developed in Shudu's lab at University of Tokyo (Miwako et al. (2007) Tamibarotene, Drugs Today (Barc) 43(8):563-568). It is a white crystalline powder with the empirical formula of C₂₂H₂₅NO₃ and the IUPAC name as 4-[(5,5,8,8-tetramethyl-5,6,7,8-tetrahydronaphthalen-2-yl)carbamoyl]benzoic acid. Tamibarotene is considered as a stable derivative of retinoic acid and the structural formula is mainly two rigid benzene rings joined by an amide bond. Tamibarotene is soluble in DMF, methanol, ethanol, DMSO, and other organic solvents. However insoluble in acetonitrile, water, and various other buffer solutions (pH3^{˜}7) (CN 101200435 B).

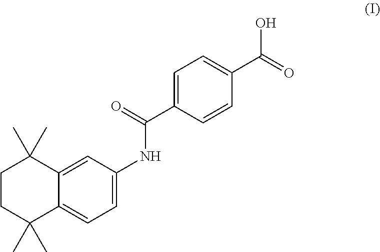 [SMILES: CC1(C)CCC(C)(C)c2cc(NC(=O)c3ccc(C(=O)O)cc3)ccc21]

Tamibarotene is a specific agonist for retinoic acid receptor alpha/beta with possible binding to retinoid X receptors (R×R). This drug is also called retinobenzoic acid and approved for treatment of relapsed and refractory acute promyelocytic leukemia in Japan in 2005 and goes under the brand name Amnolake®. (Miwako et al. (2007) Tamibarotene, Drugs Today (Barc) 43(8):563-568; “Tamibarotene:AM 80, retinobenzoic acid, Tamibaro” (2004) Drugs in R&D, 5(6):359-62). Tamibarotene was developed to overcome all trans retinoic acid (ATAR) resistance and early trials has shown it has a better tolerant than ATAR and also has shown potential antineoplastic activity against acute promyelocytic leukemia hence being investigated in possible treatment for Alzheimer's disease, multiple myeloma, Crohn's disease, (Fukasawa et al. (2012) Biological & Pharmaceutical Bulletin, 35(8):1206-1212), chronic obstructive pulmonary disease (Sakai et al. (2014) J Control Release, 196:154-160), liver cancer, and solid tumors. Tamibarotene is available as a tablet for oral suspension which contain 2 mg of free tamibarotene and the recommended dose is 6 mg/m² in two divided doses (www.Pharmacodia.com, 2012).

However, there is very little information available on manipulation of the solid forms of tamibarotene. Crystal structure of tamibarotene is published in the Cambridge Structural Database (CSD February 2017 update) (Toriumi et al. (1990) J. Org. Chem. 55:259). However, there are at least two reported crystal polymorphs of tamibarotene, polymorph 1 and polymorph 2, which have different melting points. Form 1 crystals melts at 193° C. and Form 2 crystals melts at 233° C. However, Form 1 crystals are considered extremely difficult to prepare, as there can be transitions between crystal forms with a physical impact, hence Form 1 crystals are considered unsuitable as a raw material for a pharmaceutical product. However, Form 2 crystals have higher stability, and less sensitive of heat, temperature, and light and thus has been selected for pharmaceutical drug development. (U.S. Pat. No. 8,252,837 B2)

Tamibarotene has shown favorable pharmacokinetic profile and milder side effects than ATRA in clinical trials. (Miwako et al. (2007) Tamibarotene Drugs Today (Barc) 43(8):563-568). There are additional clinical trials underway in evaluating the efficacy of tamibarotene in maintenance therapy of APL and other diseases like tumors and autoimmune diseases. Since tamibarotene is relatively, a new drug there is a larger area to study the effectiveness of tamibarotene, which would be beneficial for the development of the pharmaceutical industry. Tamibarotene has a very poor water solubility (0.000575 mg/ml, hence it is beneficial in investigating new solid forms of tamibarotene with improved solubility and subsequent bioavailability.

There is very little information available in the Cambridge Structural Database (CSD February 2017 update) on attempts, prior to this invention towards designing molecular complex of tamibarotene (tamibarotene and a cocrystal former) that would be beneficial in enhancing the physicochemical properties of the parent drug and co-former derived from. Such properties include melting point, thermal and electrical conductivity, aqueous solubility rate of dissolution, permeability, and potentially its clinical profile. It is for the first time that the concept of a molecular complex by design to assist improving the physicochemical properties of tamibarotene has been discussed here.

Adding another API to the molecular complex to form a ternary molecular complex system in a single crystalline structure is also novel, where the second API will assist or enhance the potency of the tamibarotene, would be even more advantageous in treating cancer stem cells. Thus improving the overall clinical performance of this novel ternary molecular complex.

### SUMMARY OF THE INVENTION

The present disclosure is directed towards generating new forms of tamibarotene that have improved physicochemical characteristics. One aspect of the present disclosure includes novel molecular complexes of tamibarotene neutral and ionic that includes cocrystals, salts, and solvates (e.g., hydrates and mixed solvates as well as solvates of salts), and mixtures containing such materials. In addition, the disclosure further includes methods for the preparation of such complexes.

The disclosure further includes compositions of molecular complexes of tamibarotene suitable for incorporation in a pharmaceutical dosage form. Specific molecular complexes pertaining to the disclosure include, but are not limited to, complexes of tamibarotene and gallic acid, losartan and/or telmisartan Obvious variants of the disclosed tamibarotene forms in the text, including those described by the drawings and examples will be readily apparent to the person of ordinary skill in the art having the present disclosure, and such variants are considered to be a part of the current invention.

The disclosure also includes results of characterization of the new molecular complexes by PXRD and FTIR confirming their novelty compared with that of their parent molecule and the conformer.

The foregoing and other features and advantages of the disclosed technology will become more apparent from the following detailed description, which proceeds with reference to the accompanying drawings. Such description is meant to be illustrative, but not limiting, of the invention.

### BRIEF DESCRIPTION OF THE DRAWINGS

FIG. 1. PXRD diffractograms of novel tamibarotene:gallic acid:losartan novel form.

FIG. 2. FTIR spectrum of tamibarotene:gallic acid:losartan novel form.

FIG. 3. PXRD diffractograms of novel tamibarotene:gallic acid:telmisartan novel form.

FIG. 4. FTIR spectrum of tamibarotene:gallic acid:telmisartan novel form.

FIG. 5. PXRD diffractograms tamibarotene:L-malic acid:telmisartan novel form and starting materials

FIG. 6. PXRD diffractograms tamibarotene:L-malic acid:telmisartan novel form and tamibarotene:L-malic acid (TN513) binary molecular complex

FIG. 7. PXRD of scaled up tamibarotene:L-malic acid:telmisartan novel form and its 3 months accelerated stability

FIG. 8. FTIR spectrum of tamibarotene:L-malic acid:telmisartan novel form

FIG. 9. FTIR spectrum of tamibarotene:L-malic acid:telmisartan novel form and tamibarotene:L-malic acid (TN513) binary molecular complex

FIG. 10. Sphere formation efficacy of lung cancer cells treated with ternary molecular complexes

FIG. 11. Total sphere area of lung cancer cells treated with ternary molecular complexes

### DETAILED DESCRIPTION OF THE INVENTION

In general, active pharmaceutical ingredients (APIs) in pharmaceutical compositions can be prepared in a variety of different forms. Such compounds can be prepared to have a variety of different chemical forms including chemical derivatives, solvates, hydrates, cocrystals, and/or salts. Such compounds can also be prepared to have different physical forms. For example, they may be amorphous, may have different crystalline polymorphs, or may exist in different solvated or hydrated states. The discovery of new forms of a pharmaceutically useful compound may provide an opportunity to improve the performance characteristics of a pharmaceutical product. Additionally, it expands the array of resources available for designing, for example, a pharmaceutical dosage form of a drug with a targeted release profile or other desired characteristics.

A specific characteristic that can be targeted includes the crystal form of an API. By altering the crystal form, it therefore becomes possible to vary the physical properties of the target molecule. For example, crystalline polymorphs typically have different aqueous solubility from one another, such that a more thermodynamically stable polymorph is less soluble than a less thermodynamically stable polymorph. In addition to water solubility, pharmaceutical polymorphs can also differ in properties such as rate of dissolution, shelf life, bioavailability, morphology, vapor pressure, density, color, and compressibility. Accordingly, it is desirable to enhance the properties of an active pharmaceutical compound by forming molecular complexes such as a cocrystal, a salt, a solvate or hydrate with respect to aqueous solubility, rate of dissolution, bioavailability, Cmax, Tmax, physicochemical stability, down-stream processibility (e.g., flowability compressibility, degree of brittleness, particle size manipulation), crystallization of amorphous compounds, decrease in polymorphic form diversity, toxicity, taste, production costs, and manufacturing methods.

During the development of drugs, it is frequently advantageous to have novel crystalline forms of such drug materials that possess improved properties, including increased aqueous solubility and stability. It is also desirable, in general, to increase the dissolution rate of such solid forms and potentially increase their bioavailability if used in an oral delivery setting. This also applies to the development of novel forms of tamibarotene which, when administered orally to a subject could achieve a greater or similar bioavailability and PK profile when compared to an IV or other formulations on a dose-for-dose basis.

Cocrystals, salts, solvates, and hydrates of tamibarotene of the present invention could give rise to improved properties. For example, a new tamibarotene form is particularly advantageous if it can improve the oral bioavailability or the clinical profile of the IV version by cutting the dose for instance due to improved potency. A number of novel tamibarotene forms have been synthesized, characterized, and disclosed herein.

The techniques and approaches set forth in the present disclosure can further be used by the person of ordinary skill in the art to prepare variants thereof, said variants are considered to be part of the inventive disclosure.

The present invention further includes compositions of molecular complexes of tamibarotene, losartan; a second API widely used to treat hypertension, and a former (Gallic acid) thus forming a ternary molecular complex.

The present invention further includes compositions of molecular complexes of tamibarotene, telmisartan; a second API widely used to treat hypertension, and a former (Gallic acid) thus forming a ternary molecular complex.

The present invention further includes compositions of molecular complexes of tamibarotene suitable for incorporation in a pharmaceutical dosage form. Specific molecular complexes pertaining to the disclosure include, but are not limited to, complexes of tamibarotene and gallic acid, losartan or telmisartan, which are capable of complexing through techniques not limited to solvent evaporation of their solution in single or mixed solvent systems and slurry suspension. It is believed that adding another API would improve the overall efficacy of killing cancer stem cells and subsequent tumor treatment.

In one aspect, the invention provides for a molecular complex of tamibarotene and a former selected from the group consisting of: gallic acid, losartan, or telmisartan. In one embodiment, the molecular complex is a crystalline form of tamibarotene and a former selected from the group consisting of: gallic acid, losartan, and telmisartan. Crystalline forms between tamibarotene and a former, e.g., cocrystal former, are denoted using a “:” between tamibarotene and the name of the former, i.e., tamibarotene:“former”, as well as the second API.

In one embodiment, the crystalline form is a tamibarotene:gallic acid:telmisartan crystalline form. In one embodiment, the crystalline form of tamibarotene:gallic acid:telmisartan is a 1:1:1 complex. In another embodiment, the tamibarotene:gallic acid:telmisartan crystalline form is a co-crystal. In one embodiment, the tamibarotene:gallic acid:telmisartan crystalline form is characterized by a powder X-ray diffraction peaks selected from about 3.5, 22.0, and 26.5° 2Θ±0.2° 2Θ. In one embodiment tamibarotene:gallic acid:telmisartan crystalline form is characterized by a powder X-ray diffraction peaks comprising a powder X-ray diffraction peak at about 3.5° 2Θ±0.2° 2Θ. In another embodiment tamibarotene:gallic acid:telmisartan crystalline form is characterized by a powder X-ray diffraction peaks comprising a powder X-ray diffraction peak at about 22.0° 2Θ±0.2° 2Θ. In another embodiment tamibarotene:gallic acid:telmisartan crystalline form is characterized by a powder X-ray diffraction peaks comprising a powder X-ray diffraction peak at about 26.5° 2Θ±0.2° 2Θ. In another embodiment tamibarotene:gallic acid:telmisartan crystalline form is characterized by a powder X-ray diffraction peaks comprising any two powder X-ray diffraction peaks selected from about 3.5, 22.0, and 26.5° 2Θ0.2° 2Θ. In another embodiment tamibarotene:gallic acid:telmisartan crystalline form is characterized by a powder X-ray diffraction peaks comprising powder X-ray diffraction peaks selected from about 3.5, 22.0 and 26.5° 2Θ±0.2° 2Θ.

In one embodiment, the crystalline form is a tamibarotene:gallic acid:losartan crystalline form. In another embodiment, the crystalline form of tamibarotene:gallic acid:losartan is a 1:1:1 complex. In yet another embodiment, the tamibarotene:gallic acid:losartan crystalline form is a co-crystal. In a further embodiment, The tamibarotene:gallic acid:losartan crystalline form is characterized by a powder X-ray diffraction peaks selected from about 3.5, 10.5, 19.5, 21.5, and 26.5° 2Θ±0.2° 2Θ.

In one embodiment tamibarotene:gallic acid:losartan crystalline form is characterized by a powder X-ray diffraction peaks comprising a powder X-ray diffraction peak at about 3.5° 2Θ±0.2° 2Θ. In another embodiment tamibarotene:gallic acid:losartan crystalline form is characterized by a powder X-ray diffraction peaks comprising a powder X-ray diffraction peak at about 10.5° 2Θ±0.2° 2Θ. In another embodiment tamibarotene:gallic acid:losartan crystalline form is characterized by a powder X-ray diffraction peaks comprising a powder X-ray diffraction peak at about 19.5° 2Θ±0.2° 2Θ. In another embodiment tamibarotene:gallic acid:losartan crystalline form is characterized by a powder X-ray diffraction peaks comprising a powder X-ray diffraction peak at about 21.5° 2Θ±0.2° 2Θ. In another embodiment tamibarotene:gallic acid:losartan crystalline form is characterized by a powder X-ray diffraction peaks comprising a powder X-ray diffraction peak at about 26.5° 2Θ±0.2° 2Θ. In another embodiment tamibarotene:gallic acid:losartan crystalline form is characterized by a powder X-ray diffraction peaks comprising any four powder X-ray diffraction peaks selected from about 3.5, 10.5, 19.5, 21.5, or 26.5° 2Θ±0.2° 2Θ. In another embodiment tamibarotene:gallic acid:losartan crystalline form is characterized by a powder X-ray diffraction peaks comprising any three powder X-ray diffraction peaks selected from about 3.5, 10.5, 19.5, 21.5, or 26.5° 2Θ±0.2° 2Θ. In another embodiment tamibarotene:gallic acid:losartan crystalline form is characterized by a powder X-ray diffraction peaks comprising any two powder X-ray diffraction peaks selected from about 3.5, 10.5, 19.5, 21.5, or 26.5° 2Θ±0.2° 2Θ. In another embodiment tamibarotene:gallic acid:losartan crystalline form is characterized by a powder X-ray diffraction peaks comprising powder X-ray diffraction peaks selected from about 3.5, 10.5, 19.5, 21.5 and 26.5° 2Θ±0.2° 2Θ.

In another embodiment, the crystalline form is a tamibarotene:L-malic acid:telmisartan crystalline form. In one embodiment, the crystalline form of tamibarotene:L-malic acid:telmisartan is a 1:1:1 complex. In another embodiment, the tamibarotene:L-malic acid:telmisartan crystalline form is a co-crystal. In one embodiment, the tamibarotene:L-malic acid:telmisartan crystalline form is characterized by a powder X-ray diffraction peaks selected from about 6.4, 7.0, 14.5, 16.0 and 23.4° 2Θ±0.2° 2Θ. In one embodiment tamibarotene:L-malic acid:telmisartan crystalline form is characterized by a powder X-ray diffraction peaks comprising a powder X-ray diffraction peak at about 6.4° 2Θ±0.2° 2Θ. In another embodiment tamibarotene:L-malic acid:telmisartan crystalline form is characterized by a powder X-ray diffraction peaks comprising a powder X-ray diffraction peak at about 7.0° 2Θ±0.2° 2Θ. In another embodiment tamibarotene:L-malic acid:telmisartan crystalline form is characterized by a powder X-ray diffraction peaks comprising a powder X-ray diffraction peak at about 14.5° 2Θ±0.2° 2Θ. In one embodiment, the tamibarotene:L-malic acid:telmisartan crystalline form is characterized by a powder X-ray diffraction peaks selected from about 16.0° 2Θ±0.2° 2Θ. In one embodiment, the tamibarotene:L-malic acid:telmisartan crystalline form is characterized by a powder X-ray diffraction peaks selected from about 23.4° 2Θ0.2° 2Θ. In another embodiment tamibarotene:L-malic acid:telmisartan crystalline form is characterized by a powder X-ray diffraction peaks comprising any two powder X-ray diffraction peaks selected from about 3.5, 22.0, and 26.5° 2Θ±0.2° 2Θ. In another embodiment, the tamibarotene:L-malic acid:telmisartan crystalline form is characterized by a powder X-ray diffraction peaks selected from about 6.4, 7.0, 14.5, 16.0 and 23.4° 2Θ±0.2° 2Θ.

The present invention includes complexes of tamibarotene malic acid, and gallic acid, losartan or telmisartan, which are capable of complexing in the solid-state for example, through dry or solvent-drop grinding, heating or solvent evaporation of their solution in single or mixed solvent systems, slurry suspension, antisolvent, supercritical fluids or other techniques known to a person skilled in the art. Solvents and antisolvents used to make the crystalline forms include acetone, ethanol, methanol, ethylacetate (EtOAc), isopropanol (IPA), or isopropylacetate (IPAc), diethoxymethane (DEM), Toluene, BuOAc, N-methylpyrrolidone (NMP), and heptane.

In one embodiment, the invention includes crystalline forms of tamibarotene L-malic acid and gallic acid, losartan or telmisartan, which are capable of complexing through solvent evaporation of their solution in single or mixed solvent systems, and slurry suspension.

In another aspect, the invention provides for a pharmaceutical composition comprising a molecular complex of the present invention. In one embodiment, the molecular complex is a ternary molecular complex. In another embodiment, the ternary molecular complex is crystalline form. In a further embodiment, the crystalline form is a crystalline form of tamibarotene L-malic and gallic acid, losartan or telmisartan. In another embodiment, the crystalline form is a cocrystal of tamibarotene and gallic acid, losartan or telmisartan.

In another aspect, the invention provides for a pharmaceutical composition comprising a molecular complex of the present invention and a chemotherapeutic agent. In one embodiment, the molecular complex is a ternary molecular complex and a chemotherapeutic agent. In another embodiment, the ternary molecular complex and the chemotherapeutic agent are in crystalline form. In a further embodiment, the crystalline form is a crystalline form of tamibarotene L-malic and gallic acid, losartan or telmisartan mixed with a chemotherapeutic agent. In another embodiment, the crystalline form is a cocrystal of tamibarotene and gallic acid, losartan or telmisartan mixed with a chemotherapeutic agent.

A chemotherapeutic agent is a chemical compound that has been known to treat cancer, either directly or indirectly by inhibiting the rapid proliferation of cancer cells. They include mono and bifunctional alkylators, anthracyclines, cycloskeletal disruptors, epothilones, histone deacetyl inhibitors, topoisomerase I and II inhibitors, kinase inhibitors, nucleotide and precursor analogs, peptide inhibitors, platinum-based inhibitors, retinoids, vinca alkaloids and their derivatives.

The pharmaceutical composition comprises a therapeutically effective amount of at least one of the novel molecular complexes of tamibarotene according to the invention and at least one pharmaceutically acceptable excipient. The term “excipient” refers to a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for intravenous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

The pharmaceutical composition comprises a therapeutically effective amount of at least one of the novel molecular complexes of tamibarotene according to the invention and a chemotherapeutic agent and at least one pharmaceutically acceptable excipient. The term “excipient” refers to a pharmaceutically acceptable, inactive substance used as a carrier for the pharmaceutically active ingredient(s) and includes antiadherents, binders, coatings, disintegrants, fillers, diluents, flavors, bulkants, colours, glidants, dispersing agents, wetting agents, lubricants, preservatives, sorbents and sweeteners. The choice of excipient(s) will depend on factors such as the particular mode of administration and the nature of the dosage form. Solutions or suspensions used for intravenous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

In one embodiment of the present invention, the chemotherapeutic agent is administered prior to the administration of the ternary molecular complex. The chemotherapeutic agent may be administered on a single day or be administered on several days prior to the ternary molecular complex.

In another embodiment of the invention, the chemotherapeutic agent is administered following the administration of the ternary molecular complex. In this embodiment, the chemotherapeutic agent may be administered once or several times prior to the ternary molecular complex. This administration may take place on a single day or on a series of days prior to the administration of the ternary molecular complex.

In another embodiment of the invention, the chemotherapeutic agent is administered prior to and subsequent to the administration of the ternary molecular complex. This administration may take place on one or multiple days prior to and one or multiple days subsequent to the administration of the ternary molecular complex.

The active pharmaceutical ingredient (API) (novel ternary complex and the chemotherapeutic agent) of the present invention may be in any pharmaceutical dosage form. The pharmaceutical formulation of the API may be, for example, a tablet, capsule, nanoparticulate material, e.g., granulated particulate material or a powder, a lyophilized material for reconstitution, liquid suspension, injectable suspension or solution, suppository, or topical or transdermal preparation or patch. The API generally contain about 1% to about 99% by weight of at least one novel molecular complex of tamibarotene of the invention and 99% to 1% by weight of a suitable chemotherapeutic agent.

A pharmaceutical formulation of the API of the present invention may be in any pharmaceutical dosage form. The pharmaceutical formulation may be, for example, a tablet, capsule, nanoparticulate material, e.g., granulated particulate material or a powder, a lyophilized material for reconstitution, liquid suspension, injectable suspension or solution, suppository, or topical or transdermal preparation or patch. The pharmaceutical formulations generally contain about 1% to about 99% by weight of the API and 99% to 1% by weight of a suitable pharmaceutical excipient. In one embodiment, the dosage form is an oral dosage form. In another embodiment, the dosage form is a parenteral dosage form. In one embodiment, the pharmaceutical dosage form is a unit dose. The term “unit dose” refers to the amount of API administered to a patient in a single dose.

The novel molecular complexes of tamibarotene are therapeutically useful for the treatment and/or prevention of a disease for which it is indicated, e.g., cancer. Accordingly, in another aspect, the invention also relates a method of treating or preventing a disease for which tamibarotene is indicated, said method comprising the step of administering to a patient in need thereof, a therapeutically effective amount of a pharmaceutical composition of the present invention.

In some embodiments, a pharmaceutical composition of the present invention is delivered to a subject via intratumoral injection. “Intratumoral injection” is a route of administration by which a pharmaceutical composition is delivered directly to the tumor via an injection device (e.g., needle and syringe). In other embodiments, a pharmaceutical composition of the present invention is delivered to a subject via a parenteral route, an enteral route, or a topical route.

Examples of parental routes the present invention include, without limitation, any one or more of the following: intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracoronary, intracorporus, intracranial, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intraocular, intrasinal, intraspinal, intrasynovial, intratendinous, intratesticular, intrathecal, intrathoracic, intratubular, intratumoral, intratympanic, intrauterine, intravascular, intravenous (bolus or drip), intraventricular, intravesical, and/or subcutaneous.

Enteral routes of administration of the present invention include administration to the gastrointestinal tract via the mouth (oral), stomach (gastric), and rectum (rectal). Gastric administration typically involves the use of a tube through the nasal passage (NG tube) or a tube in the esophagus leading directly to the stomach (PEG tube). Rectal administration typically involves rectal suppositories.

Topical, including transdermal, routes of administration of the present invention include administration to a body surface, such as skin or mucous membranes. Delivery vehicles of the present disclosure may be administered topically (or transdermally) via a cream, foam, gel, lotion or ointment, for example.

As used herein, the terms “treat,” “treating,” or “treatment” means to alleviate, reduce, or abrogate one or more symptoms or characteristics of a disease and may be curative, palliative, prophylactic or slow the progression of the disease. The term “therapeutically effective amount” is intended to mean that amount of drug that will elicit a desired biological or pharmacological response, i.e., an amount sufficient to treat said disease. The term “patient” includes mammals, especially humans. In one embodiment, the patient is a human. In another embodiment, the patient is a human male. In another embodiment, the patient is a human female.

In one embodiment, the invention provides for a method of treating pre-cancer or cancer comprising the step of administering to a cancer patient a therapeutically effective amount of a pharmaceutical composition of the present invention. The present invention further provides for a medicament comprising a pharmaceutical composition of the present invention for use in treating pre-cancer or cancer.

The dosage may vary depending upon the dosage form employed, sensitivity of the patient, and the route of administration. Dosage and administration are adjusted to provide sufficient levels of the active agent(s) or to maintain the desired effect. Factors, which may be taken into account, include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In some embodiments, the cancer is selected from: Wilms' tumor, rhabdomyosarcoma, ovarian cancer (e.g., germ cell), gestational trophoblastic neoplasm, Ewing's sarcoma, metastatic testicular tumors (e.g., nonseminoatous), gestational trophoblastic neoplasm, locally recurrent or locoregional solid tumors (sarcomas, carcinomas and adenocarcinomas), acute myeloid leukemia (AML), prostate cancer, skin cancer, actinic keratosis, Bowen's disease, adjuvant cancer therapy or neoadjuvant cancer therapy. In a preferred embodiment, the cancer is skin cancer, actinic keratosis, or Bowen's disease. In a further embodiment, the skin cancer is selected from the group consisting of: basal cell carcinoma (BCC), squamous cell carcinoma (SCC), and melanoma. In another embodiment, the cancer is prostate cancer. In a further embodiment, the prostate cancer is selected from the group consisting of: acinar adenocarcinoma, ductal adenocarcinoma, transitional cell (or urothelial) cancer, squamous cell cancer, small cell prostate cancer, carcinoid, and sarcoma.

### EXAMPLES

The techniques and approaches set forth in the present disclosure can further be used by the person of ordinary skill in the art to prepare variants thereof, said variants are considered to be part of the present invention.

Materials used to create the novel forms of the present inventions are commercially available and means to synthesize them as well known. Tamibarotene as a starting material used in all experiments in this disclosure was supplied by Selleck Chemicals Inc., Houston, Tex., USA with >99% purity. All other pure chemicals (Analytical Grade) were purchased form available commercial sources and used as purchased.

Solid Phase Characterization

Analytical techniques used to observe the crystalline forms include powder X-ray diffraction (PXRD) and Fourier transform infrared spectroscopy (FTIR). The particular methodology used in such analytical techniques should be viewed as illustrative, and not limiting in the context of data collection. For example, the particular instrumentation used to collect data may vary; routine operator error or calibration standards may vary; sample preparation method may vary (for example, the use of the KBr disk or Nujol mull technique for FTIR analysis).

Fourier Transform FTIR Spectroscopy (FTIR): FTIR analysis was performed on a Perkin Elmer Spectrum 100 FTIR spectrometer equipped with a solid-state ATR accessory.

Powder X-Ray Diffraction (PXRD): All tamibarotene novel molecular complex products were observed by a D-8 Bruker X-ray Powder Diffractometer using Cu Kα (λ=1.540562 Å), 40 kV, 40 mA. The data were collected over an angular range of 3° to 40° 2Θ in continuous scan mode at room temperature using a step size of 0.037° 2Θ and a scan speed of 6.17°/min.

The following examples illustrate the invention without intending to limit the scope of the invention.

### Example 1: Preparation of Tamibarotene:Gallic Acid:Losartan Complex

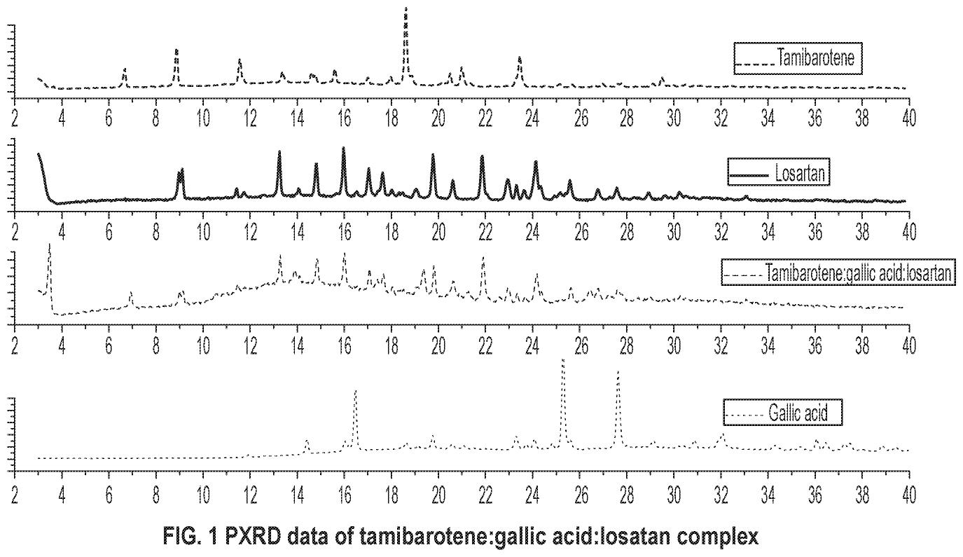

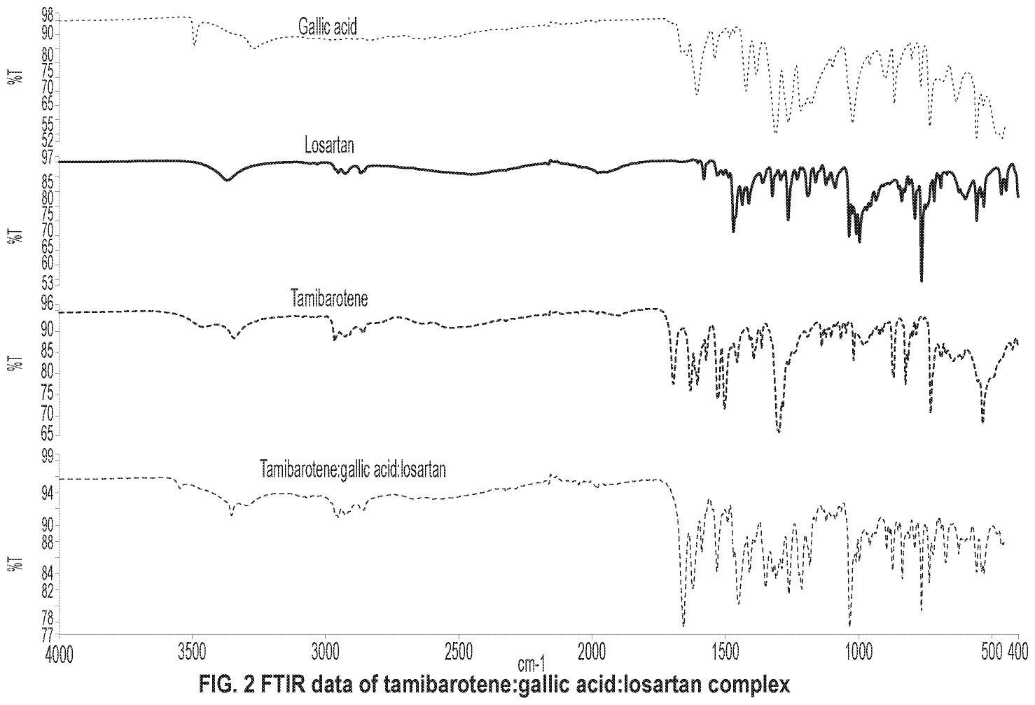

30 mg of recrystallized tamibarotene in acetonitrile and 14.5 mg of gallic acid and 36 mg (1:1:1 molar ratio) was stirred as a slurry in an open 20 mL glass vial with 1 mL of acetone. After 12-16 hours the stirring was stopped, and the mixture was dried at room temperature for another 12-16 hours. The solids gathered were dried and stored in a screw cap vials for subsequent analysis. The material was characterized by PXRD and FTIR corresponding to FIGS. 1 and 2, respectively.

### Example 2: Preparation of Tamibarotene:Gallic Acid:Telmisartan Complex

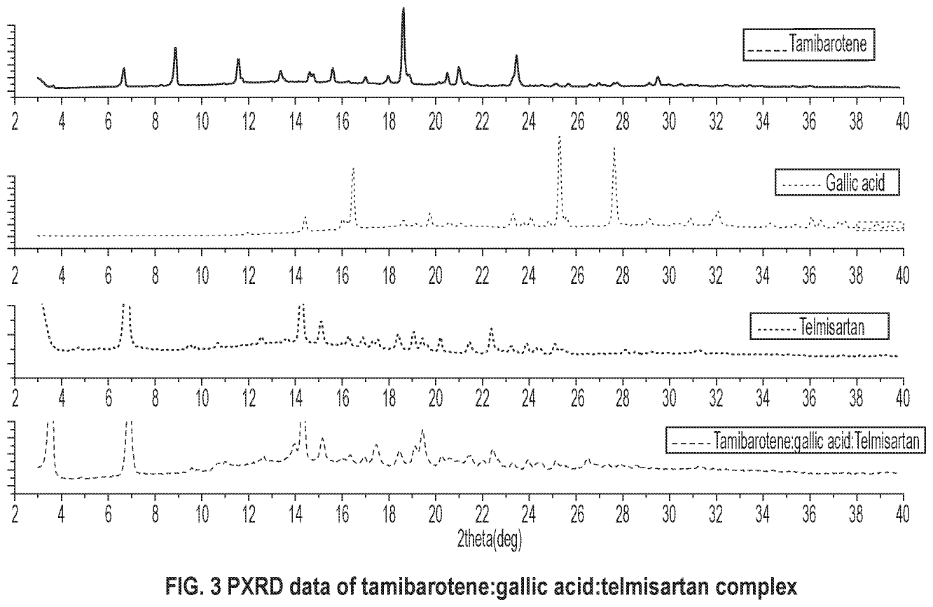

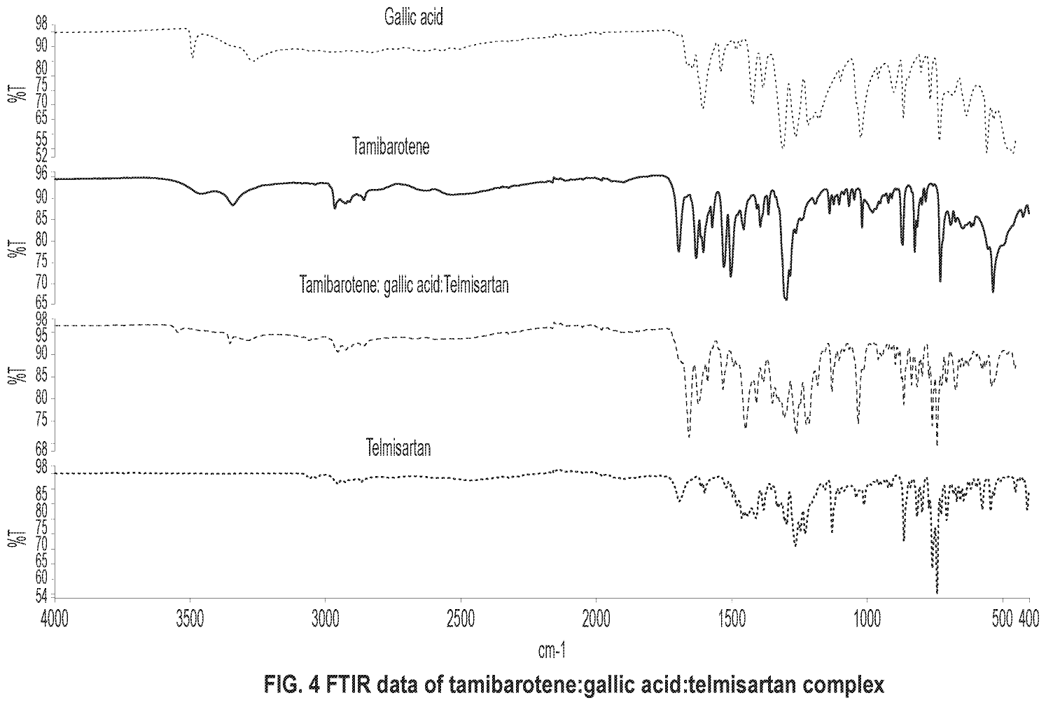

30 mg of recrystallized tamibarotene in acetonitrile and 14.5 mg of gallic acid and 44 mg of telmisartan (1:1:1 molar ratio) was stirred as a slurry in an open 20 mL glass vial with 1 mL of acetone. After 12-16 hours the stirring was stopped, and mixture was dried at room temperature for another 12-16 hours. Solids were dried and stored in a screw cap vials for subsequent analysis. All materials were characterized by PXRD and FTIR corresponding to FIGS. 3 and 4, respectively.

### Example 3: Preparation of Tamibarotene:L-Malic Acid:Telmisartan Molecular Complex

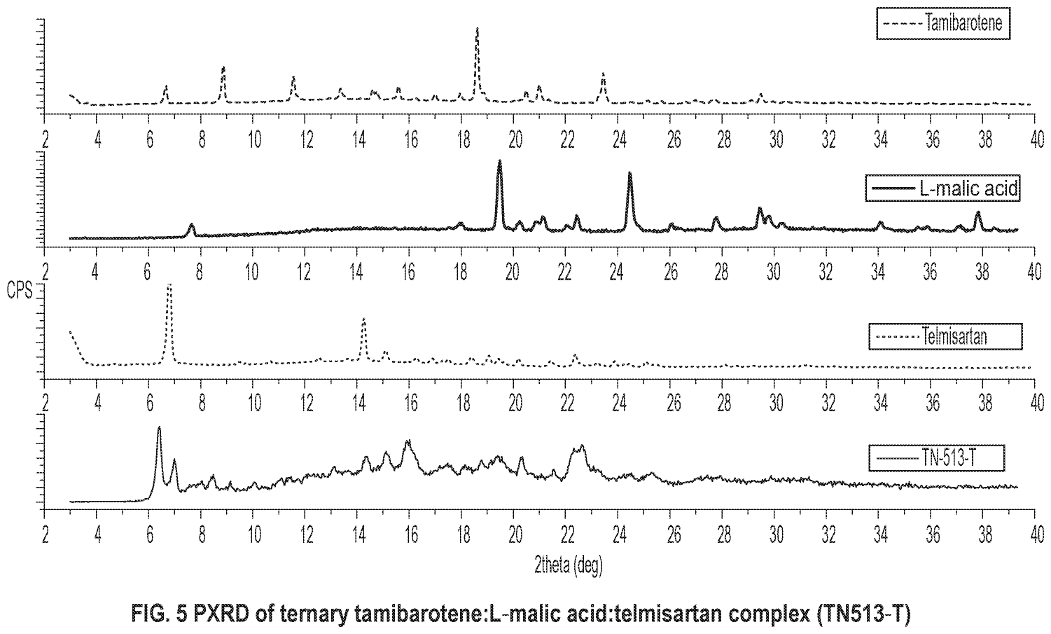

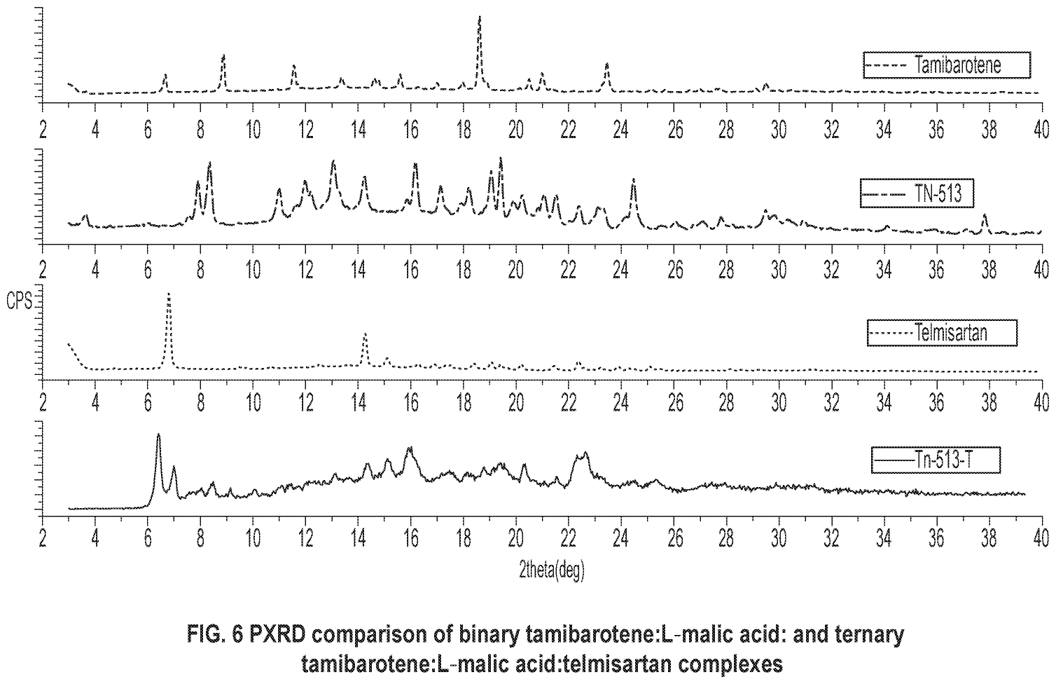

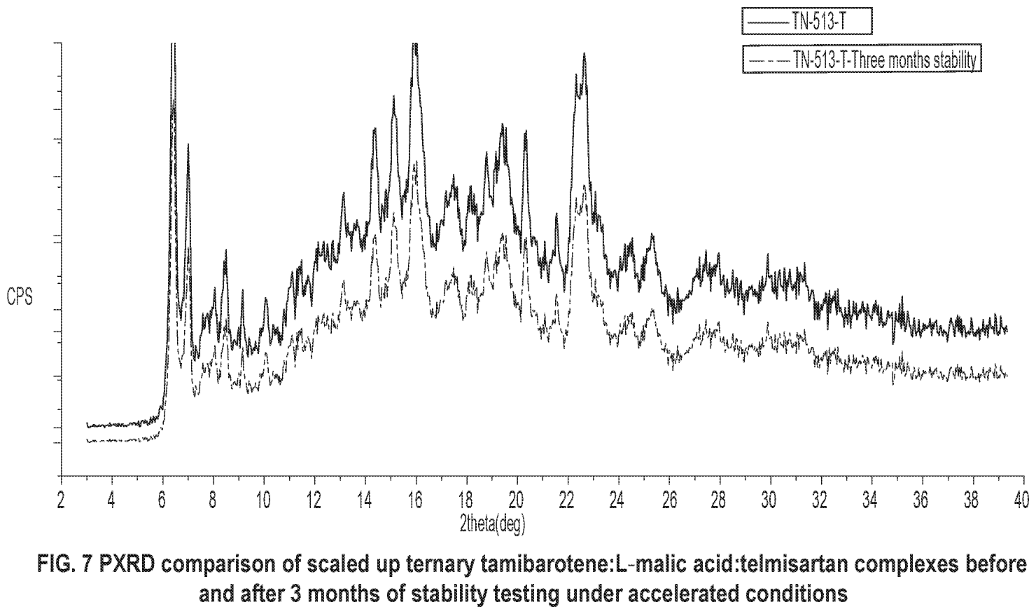

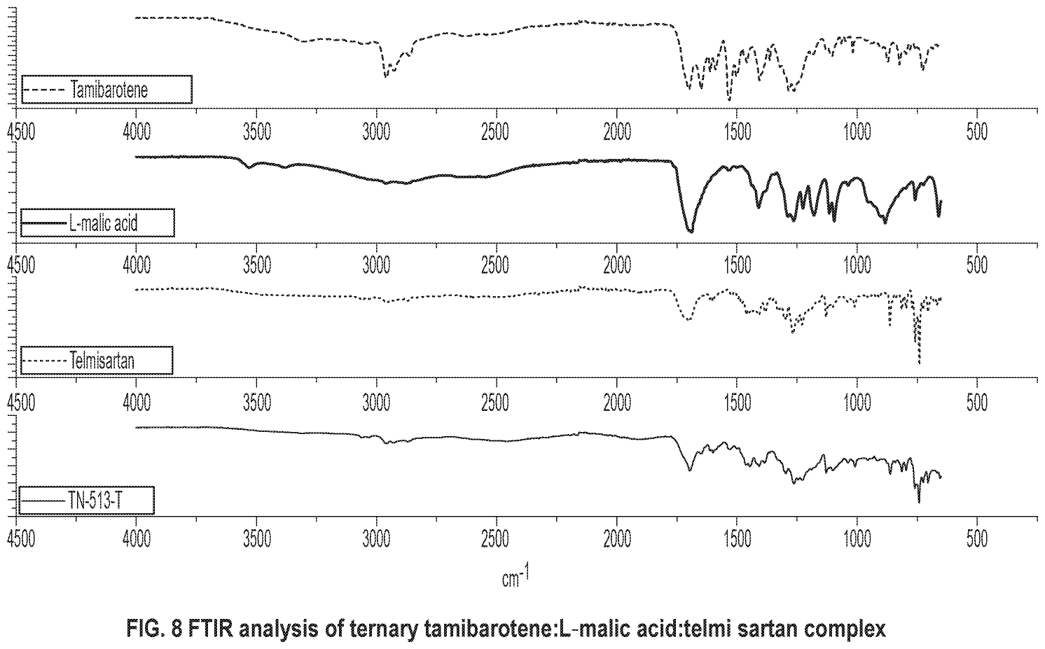

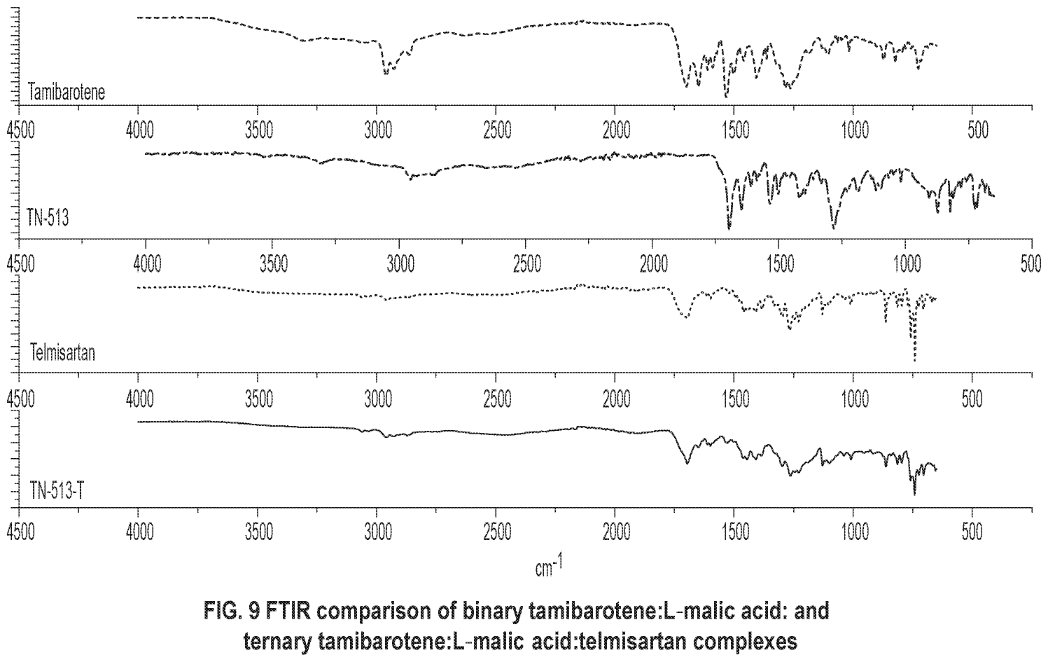

30.0 mg of tamibarotene, 11.4 mg of L-malic acid and 43.5 mg of telmisartan (1:1:1 molar ratio) were stirred as an open slurry in 1 mL of acetone in a glass vial. After 16-24 hours of stirring mixture was further dried at room temperature until, a completely dried mixture is obtained. The resulted material was stored in a screw cap vial and characterized by PXRD and IR. See FIGS. 5, 6, 8, and 9. PXRD comparison of binary tamibarotene-L-malic acid complex (TN-513) with tamibarotene-L-malic acid-telmisartan (TN-513-T) also confirmed the successful synthesis of a ternary complex. Synthesis of ternary TN-513-T was scaled up to 150 mg of tamibarotene and stability was tested on accelerated conditions (75% humidity at 40° C.) for 1 and three months. Both scale up and stability studies confirmed that there is no change in PXRD data compared to the initial synthesis of TN-513-T. See FIG. 7.

### Example 4: IC₅₀ Studies

All IC₅₀ studies' methodology and materials are described in full details in international publication number WO 2019/160806 dated Aug. 22, 2019, but with substituting the binary molecular complex with the ternary molecular complex of the present invention. The results of these studies suggest that ternary molecular complexes have mare potency in killing cancer cells than the control (pure tamibarotene). It also showed that the ternary molecular complex with Telmisartan has consistently showed better efficacy with several types of cancer compared to other sartans as shown in Table 1. Surprisingly, that efficacy was much enhanced with the addition of small amounts of widely used chemotherapeutic agents (e.g., paclitaxel and 5-fluorouracil) to such a degree test results in colon cancer cell lines for example were below the limits of detection (<<0.1) on the micromole scale level as shown in Table 2.

TABLE 1
IC₅₀ data of two averaged triplicates for the ternary molecular complexes.

| Monolayer Cell Culture Assay | | | | | |
|---|---|---|---|---|---|
| Cancer | | | IC₅₀ Test(I) | IC₅₀ Test (II) | IC₅₀ Test AVG |
| Type | Cell Line | Compounds | Avg (μM) | Avg (μM) | Avg (μM) |
| Sarcoma | SK-ES-1 | TAM | >>100 | >>100 | >>100 |
| | SK-ES-1 | TN513:CS | 29.16 | 33.58 | 31.37 |
| | SK-ES-1 | TN513:LS | 90.01 | 122.1 | 106.05 |
| | SK-ES-1 | TN513:TS | 17.45 | 18.39 | 17.92 |
| Sarcoma | U-2 OS | TAM | >>100 | >>100 | >>100 |
| | U-2 OS | TN513:CS | 39.88 | 45.79 | 42.8 |
| | U-2 OS | TN513:LS | 59.87 | 69.63 | 64.75 |
| | U-2 OS | TN513:TS | 11.21 | 14.58 | 12.90 |
| Skin | SK-Mel-5 | TAM | >>100 | >>100 | >>100 |
| | SK-Mel-5 | TN513:CS | 49.51 | 42.63 | 46.07 |
| | SK-Mel-5 | TN513:LS | 99.90 | 132.2 | 116.1 |
| | SK-Mel-5 | TN513:TS | 15.33 | 26.10 | 20.71 |
| Prostate | PC-3 | TAM | >>100 | >>100 | >>100 |
| | PC-3 | TN513:CS | 101.8 | 110.8 | 106.3 |
| | PC-3 | TN513:LS | 321.5 | 342.6 | 332.1 |
| | PC-3 | TN513:TS | 71.72 | 70.76 | 71.24 |
| Lung | A549 | TAM | 59.90 | 60.64 | 60.27 |
| | A549 | TN513:CS | 42.70 | 62.23 | 52.4 |
| | A549 | TN513:LS | 93.79 | 126.2 | 110.0 |
| | A549 | TN513:TS | 11.97 | 19.95 | 15.96 |
| Pancreas | PANC-1 | TAM | >>100 | >>100 | >>100 |
| | PANC-1 | TN513:CS | 53.26 | 88.01 | 70.63 |
| | PANC-1 | TN513:LS | 103.8 | 149.0 | 126.4 |
| | PANC-1 | TN513:TS | 24.82 | 40.18 | 32.5 |

TAM = Pure Tamibarotene,
TN513:CS = Tamibarotene:L-malic acid:candesartan,
TN513:LS = Tamibarotene:L-malic acid:losartan,
TN513:TS Tamibarotene:L-malic acid:telmisartan

TABLE 2
IC₅₀ data of two averaged triplicates for the ternary molecular complexes.

| Monolayer Cell Culture Assay | | | | | |
|---|---|---|---|---|---|
| Cancer | | | IC₅₀ Test (I) | IC₅₀ Test (II) | IC₅₀ Test AVG |
| Type | Cell Line | Compounds | Avg (μM) | Avg (μM) | Avg (μM) |
| Colon | CT26 | TAM | 61.89 | 94.75 | 87.32 |
| | CT26 | TN513:TS + 2 UM 5FU | 0.22 | 0.78 | 0.5 |
| | CT26 | TN513:TS + 10 UM 5FU | <<0.1 | <<0.1 | <<0.1 |
| | MC38 | TAM | 65.69 | 136.8 | 101.24 |
| | MC38 | TN513:TS + 2 UM 5FU | 0.45 | 1.1 | 0.77 |
| | MC38 | TN513:TS + 10 UM 5FU | <<0.1 | <<0.1 | <<0.1 |
| | HCT116 | TAM | 49.59 | 71.27 | 60.43 |
| | HCT116 | TN513:TS + 2 UM 5FU | 4.71 | 5.60 | 5.15 |
| | HCT116 | TN513:TS + 10 UM 5FU | <<0.1 | <<0.1 | <<0.1 |
| | HT29 | TAM | 88.9 | 111.2 | 100.1 |
| | HT29 | TN513:TS + 2 UM 5FU | 16.68 | 11.91 | 14.3 |
| | HT29 | TN513:TS + 10 UM 5FU | <<0.1 | <<0.1 | <<0.1 |
| Lung | A549 | TAM | 59.90 | 60.64 | 60.27 |
| | A549 | TN513:TS | 11.97 | 19.95 | 15.96 |
| | A549 | TN513:TS + 5 nM PTX | 3.36 | 8.32 | 5.84 |
| | H460 | TN513:TS + 5 nM PTX | 1.0 | 2.75 | 1.87 |
| | H1299 | TN513:TS + 5 nM PTX | 8.37 | 8.59 | 8.47 |

TAM = Pure Tamibarotene,
TN513:TS Tamibarotene:L-malic acid:telmisartan with the addition of PTX (paclitaxel) and 5FU (5-fluorouracil)

### Example 5: Sphere Assay Studies

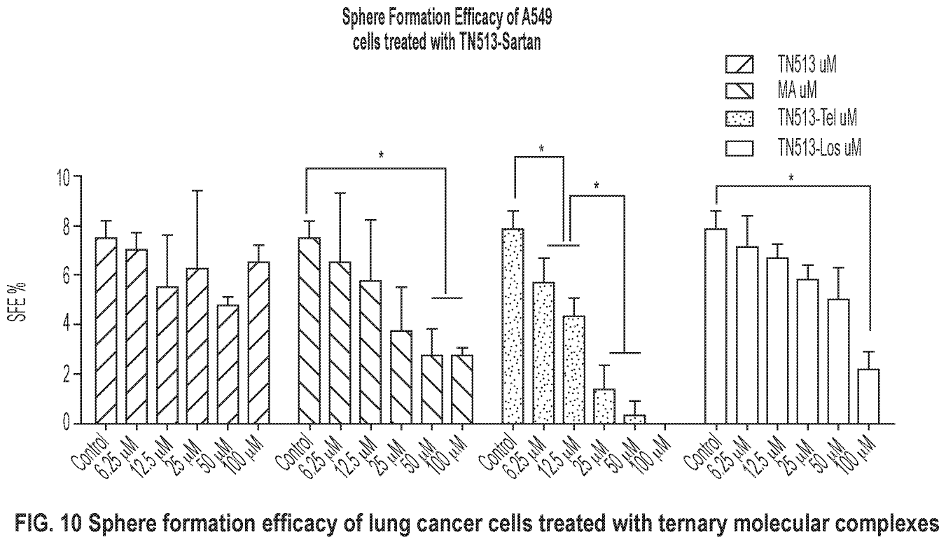

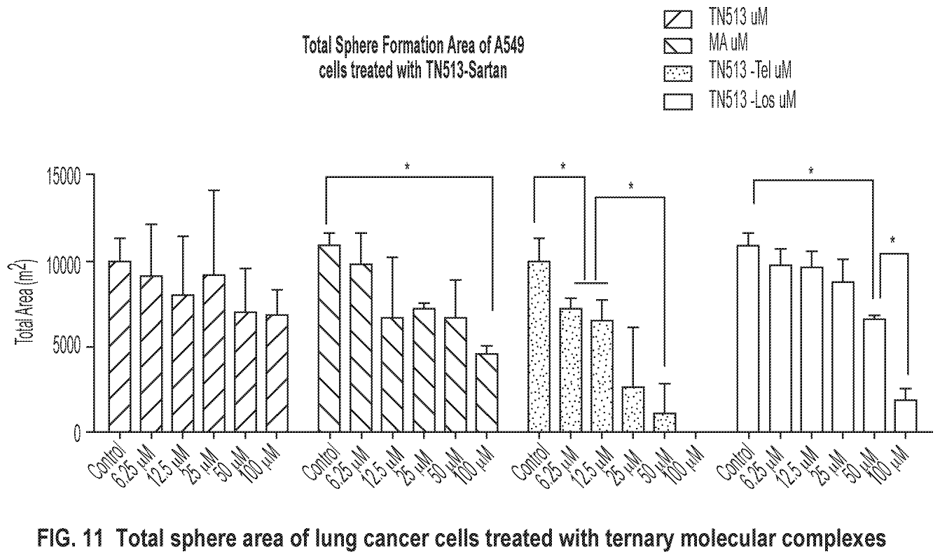

All sphere assay studies methodology and materials are described in full details in international publication number WO 2019/160806 dated Aug. 22, 2019, but with substituting the binary molecular complex with the ternary molecular complex of the present invention. The results of these studies demonstrate that the ternary molecular complex of TN513:Telmisartan has superior efficacy preventing sphere aggregation as well as dramatically reducing the total surface area of the cancer cell spheres as shown in FIGS. 10 and 11.

What is claimed:

1. A crystalline form of tamibarotene selected from the group consisting of: tamibarotene:L-malic acid:telmisartan, tamibarotene:gallic acid:losartan, and tamibarotene:gallic acid:telmisartan.

2. The crystalline form of claim 1, wherein the crystalline form is tamibarotene:L-malic acid:telmisartan.

3. The crystalline form of claim 2, wherein the crystalline form is characterized by a powder X-ray diffraction pattern comprising one or more powder X-ray diffraction peaks selected from the group consisting of: about 6.4, 7.0, 14.5, 16.0 and 23.4° 2Θ±0.2° 2Θ

4. The crystalline form of claim 1, wherein the crystalline form is tamibarotene:gallic acid:losartan.

5. The crystalline form of claim 4, wherein the crystalline form is characterized by a powder X-ray diffraction pattern comprising one or more powder X-ray diffraction peaks selected from the group consisting of: about 3.5, 10.5, 19.5, 21.5, and 26.5° 2Θ±0.2° 2Θ.

6. The crystalline form of claim 1, wherein the crystalline form is tamibarotene:gallic acid:telmisartan.

7. The crystalline form of claim 6, wherein the crystalline form is characterized by a powder X-ray diffraction pattern comprising one or more powder X-ray diffraction peaks selected from the group consisting of: about 3.5, 22.0, and 26.5° 2Θ±0.2° 2Θ.

8. A composition comprising the crystalline form of any one of claims 1-7.

9. A pharmaceutical composition comprising the crystalline form of any one of claims 1-7 and at least one pharmaceutically acceptable excipient.

10. A pharmaceutical composition comprising the crystalline form of any one of claims 1-7 and at least one chemotherapeutic agent selected from mono and bifunctional alkylators, anthracyclines, cycloskeletal disruptors, epothilones, histone deacetyl inhibitors, topoisomerase I and II inhibitors, kinase inhibitors, nucleotide and precursor analogs, peptide inhibitors, platinum based inhibitors, retinoids, vinca alkaloids and their derivatives, and a pharmaceutically accepted excipient.

11. The pharmaceutical composition of claim 9, where the pharmaceutical composition is suitable for any drug delivery route.

12. The pharmaceutical composition of claim 10, where the pharmaceutical composition is suitable for any drug delivery route.

13. The pharmaceutical composition of claim 11, wherein the pharmaceutical composition is an oral dosage form, a topical dosage form, or an injectable dosage form.

14. The pharmaceutical composition of claim 12, wherein the pharmaceutical composition is an oral dosage form, a topical dosage form, or an injectable dosage form.

15. The pharmaceutical composition of claim 13, wherein the pharmaceutical composition is a solid dosage form for reconstitution in a liquid medium.

16. The pharmaceutical composition of claim 14, wherein the pharmaceutical composition is a solid dosage form for reconstitution in a liquid medium.

17. The pharmaceutical composition of claim 15, wherein the liquid medium is an aqueous liquid.

18. The pharmaceutical composition of claim 16, wherein the liquid medium is an aqueous liquid.

19. The pharmaceutical composition of any one of claim 8-18, wherein the pharmaceutical composition is a unit dose.

20. A method of treating or preventing a disease for which tamibarotene and losartan is indicated, the method comprising the step of administering to a patient in need thereof, a therapeutically effective amount of a pharmaceutical composition of any one of claims 8-18.

21. A method of treating or preventing a disease for which tamibarotene and telmisartan is indicated, the method comprising the step of administering to a patient in need thereof, a therapeutically effective amount of a pharmaceutical composition of any one of claims 8-18.

22. The method of claim 20 or claim 21, wherein the disease is selected from: Wilms' tumor, rhabdomyosarcoma, lung, breast, colon, rectal head and neck, brain, pancreatic, ovarian cancer, gestational trophoblastic neoplasm, sarcoma, Ewing's sarcoma, metastatic testicular tumors, gestational trophoblastic neoplasm, locally recurrent or locoregional solid tumors (sarcomas, carcinomas and adenocarcinomas), acute myeloid leukemia (AML), multiple myeloma, Shwachman-Diamond syndrome, prostate cancer, skin cancer, actinic keratosis, Bowen's disease, adjuvant cancer therapy, or neoadjuvant cancer therapy.

23. A method of making the crystalline form of any one of claims 1-5, comprising the steps of: combining tamibarotene, telmisartan, and L-malic acid as a former and forming crystals of the tamibarotene, telmisartan, and the former.

24. A method of making the crystalline form of any one of claims 1-5, comprising the steps of: combining tamibarotene, losartan, and gallic acid as a former and forming crystals of the tamibarotene, losartan, and the former.

25. A method of making the crystalline form of any one of claims 1-5, comprising the steps of: combining tamibarotene, telmisartan, and gallic acid as a former and forming crystals of the tamibarotene, telmisartan, and the former.

26. The method of claim 23, wherein the method comprises the step of combining the tamibarotene, the losartan, and the former with a solvent.

27. The method of claim 24, wherein the method comprises the step of combining the tamibarotene, the telmisartan, and the former with a solvent.

28. The method of claim 25, wherein the method comprises the step of combining the tamibarotene, the telmisartan, and the former with a solvent.

29. The method of claim 23, or 24 or claim 25, wherein the solvent is selected from the group consisting of: acetone, ethanol, methanol, ethylacetate (EtOAc), isopropanol (IPA), isopropylacetate (IPAc), diethoxymethane (DEM), Toluene, BuOAc, N-methylpyrrolidone (NMP), and a heptane.
